(19) United States
(12) Patent Application Publication
(10) Pub. No.: US 2008/0269778 A1
(43) Pub. Date: Oct. 30, 2008

(54) Optically Orienting an Invasive Medical Device

(76) Inventor: Jay Waldron Patti, Boston, MA (US)

Correspondence Address: FISH & RICHARDSON PC, P.O. BOX 1022, MINNEAPOLIS, MN, 55440-1022, US

(21) Appl. No.: 11/816,021

(22) PCT Filed: Feb. 17, 2006
(86) PCT No.: PCT/US2006/005631

§ 371 Date: Mar. 25, 2008

Related U.S. Application Data
(60) Provisional application No. 60/653,962, filed on Feb. 17, 2005.

(51) Int. Cl. A61B 10/02

(52) U.S. Cl. 606/130; 600/567

(57) ABSTRACT

A method of adjusting an orientation of an apparatus relative to a surface of a sample includes positioning the apparatus in an initial orientation relative to the surface; projecting a reference pattern from the apparatus onto a reference surface, the position of the projected reference pattern on the reference surface being responsive to a change in an angular orientation of the apparatus relative to the initial orientation; on the basis of a position of the projected reference pattern determining an angular deviation of the apparatus from a desired orientation; and adjusting the orientation of the apparatus, such that the position of the reference pattern projected on the reference surface indicates a reduction in the angular deviation.

### TECHNICAL FIELD

This disclosure relates to methods and devices for orienting a device, and, more particularly, to methods and devices for optically orienting an invasive medical device.

### BACKGROUND

Minimally-invasive diagnostic and therapeutic medical procedures are becoming more prevalent with the increasing availability of imaging modalities. Although some minimally-invasive procedures use expensive imaging equipment, costs associated with minimally-invasive treatments and diagnostic procedures can be lower than alternative treatments and procedures. These cost reductions often are attributed to shorter hospital stays and decreased complications and morbidity associated with minimally-invasive procedures as compared with alternative procedures.

As imaging techniques offer more information about tissue characteristics and are able to resolve smaller structures, greater precision and accuracy is expected of imaging guided procedures. Because image-guided, minimally-invasive procedures are generally associated with shorter hospital stays for a patient, a higher proportion of the total cost of a procedure is associated with use of the imaging modality to perform the procedure. Therefore, speed, accuracy, and efficiency are desired when using expensive imaging modalities during procedures.

### SUMMARY

The invention is based on the recognition that the orientation of an instrument can be coupled to the movement of a beam from a light source associated with the instrument.

In one aspect, the invention features a method of adjusting an orientation of an apparatus relative to a surface of a sample. The method includes positioning the apparatus in an initial orientation relative to the surface; projecting a reference pattern from the apparatus onto a reference surface, the position of the projected reference pattern on the reference surface being responsive to a change in an angular orientation of the apparatus relative to the initial orientation; on the basis of a position of the projected reference pattern determining an angular deviation of the apparatus from a desired orientation; and adjusting the orientation of the apparatus, such that the position of the reference pattern projected on the reference surface indicates a reduction in the angular deviation.

Certain practices of the method include those in which projecting a reference pattern includes projecting a ring that moves in response to a change in an angular orientation of the apparatus relative to the initial orientation, and those in which projecting a reference pattern includes projecting lines that move in response to a change in an angular orientation of the apparatus relative to the orientation.

In yet other practices, projecting a reference pattern includes projecting a first beam emitted from the apparatus and a second beam emitted from the apparatus at a predetermined angle relative to the first beam.

The method can be used to adjust the orientation of a variety of different types of apparatus. For example, in some practices, positioning the apparatus includes positioning a biopsy needle.

Other practices of the method include the additional steps of inserting the apparatus into the sample; and while the apparatus is inserted, imaging the sample and the apparatus to determine the angular deviation. Among these practices are those that further include withdrawing the apparatus, at least partially, from the sample; and re-inserting the apparatus into the sample in a manner that reduces the angular deviation.

Also among these practices are those in which imaging the sample and the apparatus includes separately imaging a plurality of axial slices of the sample. In some practices, these axial slices are imaged at substantially the same phase of a periodic physiological process. Exemplary periodic physiological processes include a pulmonary cycle, and a cardiac cycle.

In another aspect, the invention features an apparatus that includes an instrument; a light source adapted for coupling to the instrument; and an optical system positioned along a path of light emitted from the light source. The optical system is adapted to transform light emitted from the light source into a reference pattern that defines a coordinate system, and to project that reference pattern on a reference surface.

Embodiments of the apparatus include those in which the instrument is a medical instrument, such as a biopsy needle.

Other embodiments include those in which the optical element is adapted to include, in the reference pattern, a feature identifying an orientation of the instrument. Exemplary features include a ring identifying an orientation of the medical instrument identifying an orientation of the instrument, and a first beam and a second beam, the second beam being oriented at a predetermined angle relative to the first beam.

A variety of light sources can be used. For example, in some embodiments, the light source includes a laser, whereas in other embodiments, the light source includes a light-emitting diode.

In other embodiments, the light source is oriented to emit light in a direction that differs from a direction defined by the instrument.

Yet other embodiments include those having an instrument guide adapted for guiding the instrument along an axis.

Another aspect features an apparatus for adjusting the angular orientation of an instrument that is adapted to be inserted into a sample. Such an apparatus includes an instrument guide adapted for guiding the instrument along an axis; a light source coupled to the instrument guide; and an optical system positioned in a path of light emitted from the light source, the optical system being adapted to transform light emitted from the light source into a reference pattern that defines a coordinate system, and to project that reference pattern onto a reference surface.

Embodiments of the foregoing apparatus include those in which the optical system is adapted to project light in a direction that differs from a direction defined by a longitudinal axis of the instrument guide.

Other embodiments of the apparatus include those in which the instrument guide is detachably coupled to the light source.

In other embodiments of the apparatus, the instrument guide includes a tube for guiding the instrument.

The new device includes a light source that displays a pattern of light that defines a coordinate system and is coupled to an instrument or apparatus that can be inserted into a sample, such as tissue in a human or animal patient. The instrument is inserted into the sample in a direction toward a target, and a deviation of the actual direction of insertion from a desired direction towards the target is determined. The coordinate system projected from the light source onto a surface is observed while the instrument is repositioned. This coordinate system is used to verify that the instrument is repositioned into the desired direction.

In another aspect, the position of an apparatus with respect to a surface of a sample is adjusted by positioning the apparatus in a first orientation with respect to the sample surface, projecting a pattern of light from the apparatus onto a display surface, where the pattern includes at least one mark identifying an angular orientation of a longitudinal axis of the apparatus with respect to the first orientation, determining an angular deviation of the longitudinal axis of the apparatus from a desired direction, and adjusting the orientation of the apparatus, such that the at least one identifying mark indicates that the longitudinal axis of the apparatus is oriented in the desired direction.

Implementations can include one or more of the following features. The pattern of light can include at least one identifying mark in the shape of a ring identifying an angular orientation of the apparatus with respect to the first orientation. The pattern of light can include lines identifying angular orientations of the apparatus with respect to the first orientation. The apparatus can include a biopsy needle. The apparatus can include a guide for a device or second apparatus. The pattern of light projected onto the surface can include a first beam of light and a second beam of light emitted from the apparatus at a predetermined angle with respect to the first beam of light. The pattern of light projected onto the surface can include a first beam of light and a second beam of light emitted from a separate apparatus or light source at a predetermined angle with respect to the first beam of light.

The apparatus can be inserted into an opaque sample through a point on the surface of the sample such that the longitudinal axis of the apparatus is aligned with the first orientation, and the sample and the apparatus can be imaged while the apparatus is inserted into the sample to determine the angular deviation of the longitudinal axis of the apparatus from the desired orientation.

The apparatus can be withdrawn at least partially from the sample and the apparatus can be re-inserted into the sample through the point on the surface of the sample, such that the longitudinal axis of the apparatus is oriented in the desired direction. A plurality of axial slices of the sample can be separately imaged. The plurality of the axial slices can be imaged at substantially the same phase during a repetitive physiological process of the sample. When the sample is a section of tissue in a living subject, such as a human or animal, the physiological process can be, for example, breathing or the beating of a heart.

In another general aspect, an apparatus for adjusting the angular orientation of an instrument that is adapted to be inserted into a sample includes an instrument, a light source fixed to the instrument or a light source with a fixed orientation with respect to the orientation of the instrument, and an optical element positioned in a path of light emitted from the light source adapted to cause light to be emitted from the light source in a pattern that defines a coordinate system.

Implementations can include one or more of the following features. For example, the instrument can be a medical instrument. The instrument can be a biopsy needle. The pattern can include at least one mark identifying an angular orientation of the instrument. The pattern of light can include at least one identifying mark in the shape of a ring identifying an angular orientation of the medical instrument. The pattern of light can include lines identifying angular orientations of the instrument. The pattern of light can include a first beam of light and a second beam of light emitted from the apparatus at a predetermined angle with respect to the first beam of light. The light source can be a laser or a light emitting diode. The light can be emitted from the light source in a direction that differs from a direction defined by a longitudinal axis of the instrument.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

Other features and advantages of the invention will be apparent from the following detailed description, and from the claims.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The words “comprising,” “including,” “having,” and other forms thereof are intended to be equivalent in meaning and to be open-ended so that an item or items following any one of these words is not meant to be an exhaustive listing of such item or items, or meant to be limited to only the listed item or items.

Other features and advantages of the invention will be apparent from the claims, the specification, and the accompanying figures, in which:

### DESCRIPTION OF DRAWINGS

FIG. 1 is schematic side view of a light source projecting a reference pattern of light onto a reference surface.

FIG. 2 is schematic side view of a light source coupled to an instrument.

FIGS. 3A to 3E are exemplary reference patterns.

FIG. 4A is a schematic three-dimensional view of an instrument inserted into a sample but oriented in a direction that leads away from a target.

FIGS. 4B-4D are sectional views of the three axial slices in FIG. 4A.

FIG. 5A is a schematic three-dimensional view of an instrument inserted into a sample towards a target within the sample.

FIG. 5B is a schematic view of the particular axial slice from FIG. 5A that includes the insertion site.

FIG. 5C is a schematic view of the particular axial slice from FIG. 5A that includes the target.

FIG. 5D is an overlay of FIGS. 5B and 5C.

FIG. 6 is a schematic view of an axial slice of a sample though which an instrument penetrates.

FIG. 7 is a schematic view of a reference pattern projected from a light source onto a reference surface that includes a reference mark.

FIG. 8 is a schematic three-dimensional view of an instrument guide coupled to a light source housing.

FIG. 9 is a schematic view of an instrument being guided through a sample to reach a target beyond the other side of the sample.

### DETAILED DESCRIPTION

A limitation of minimally-invasive procedures is the inability to control bleeding if major vascular structures are breached. Thus, when target areas are located close to large vascular, or other vital structures, operators must have sufficient technical expertise to avoid such structures, while still reaching the target area.

An imaging-guided, invasive or minimally-invasive procedure on a patient using the new methods and systems can involve obtaining multiple cross-sectional axial images of the area of interest in relation to a point or grid of fiduciary markers placed over the estimated area of entry into the patient. The images are then analyzed, and an insertion site is chosen. After marking the insertion site on the patient's skin, an instrument is partially inserted into the patient from the insertion site along a direction that is expected to intersect the target. The angular orientation of the instrument in its partially inserted position is noted by projecting a laser beam whose path is related to the orientation of the instrument. The beam projects a reference pattern onto a reference surface, such as a wall or ceiling in the operating room. There is at least one fixed reference mark, such as a spot, on the reference surface.

After the instrument is partially inserted towards the target, the patient can be re-imaged to determine the accuracy of the initial insertion direction, and an angular difference between an axis of the instrument and a line defined by the entry point and the target can be calculated. Using information from the re-image and the calculated angular difference, the position and direction of the instrument (or a new instrument placed alongside the first instrument) can be adjusted with a freehand technique approximately onto the line defined by the insertion site and target. The angular position of the instrument can be verified by observing the change in position of the projected reference pattern relative to the fixed reference point on the reference surface within the operating room. After the angular position of the instrument is adjusted, the instrument can be advanced towards the target while confirming the new alignment by again observing the laser pattern relative to the fixed reference point. The re-imaging and re-positioning steps are repeated until the tip of the instrument is at the target.

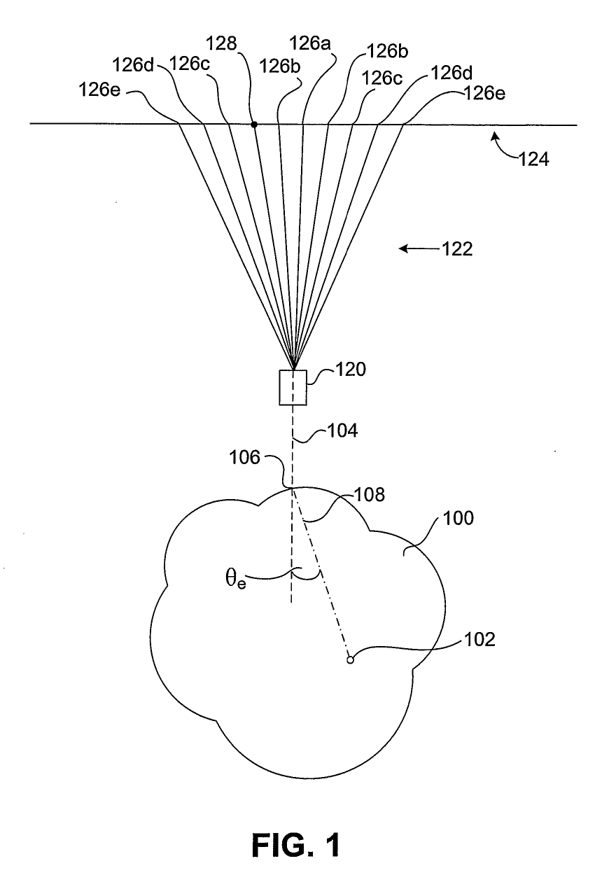

Referring to FIG. 1, a sample 100 can include a target 102 that an operator wishes to reach with an instrument 104 (e.g., a needle, a probe, or a drill). The instrument 104 is inserted into the sample 100 at an insertion site 106 on the surface of the sample 100 in the general direction of the target 102. The operator attempts to insert the instrument 104 along a line 108 extending from the insertion site 106 to the target 102 to reach the target. However, in many cases, a path 110 of a longitudinal axis of the instrument 104 deviates from the desired line 108 by an angle, θₑ.

A light source 120 (e.g., a laser, a light emitting diode (“LED”), an incandescent lamp, or other light) coupled to the instrument 104 emits light that is ultimately projected in a reference pattern 122 (e.g., a coordinate system) that is displayed on a reference surface 124 in the environment in which the instrument inserting procedure occurs. This reference pattern 122 includes a feature responsive to changes in angular orientation of the instrument 104.

For example, the reference pattern 122 can include a series of lines or rings emitted from the light source 120 in directions that deviate from an axial direction of the instrument 104 by known angles. Thus, when projected on the fixed surface 124, a spot 126a indicates a direction that is aligned with the axis of the instrument 104; an innermost light ring 126b centered on the spot 126a indicates directions that deviate from the axis of the instrument 104 by, for example, one degree; further concentric light rings 126c, 126d, and an outermost light ring 126e indicate directions that deviate from the axis of the instrument 104 by progressively greater angles, for example, two, three, and four degrees respectively. The projected pattern 122, therefore, creates a coordinate system that is fixed relative to a longitudinal axis of the instrument 104, such that when the instrument 104 moves or changes orientation the projected pattern 122 on the nearby reference surface 124 also moves.

To change the orientation of the instrument 104 by a desired amount, one compares the position of the reference pattern 122 with the position of a reference mark 128 on the reference surface 124. For example, when the instrument 104 is inserted into the sample 100 in an initial orientation, the left side of the innermost light ring 126b can be projected onto the reference mark 128. Then, to change the angular orientation of the instrument 104 by, for example, five degrees, the instrument 104 is withdrawn from the sample 104, at least partially, and reinserted in a second orientation in which the right side of the outermost light ring 126e is projected onto the reference mark 128. Because the right side of the outermost light ring 126e is emitted from the light source 120 at an angle that differs by five degrees from the left side of the light ring 126b, changing the alignment of the instrument 104 from a position in which the left side of the light ring 126b is projected onto the reference mark 128 to a position in which the right side of the light ring 126e is projected onto the reference mark 128 indicates that the alignment of the instrument 104 has been changed by five degrees. In practice, because the distance from the light source 120 to the fixed surface 124 is much greater than the axial displacement of the light source 120 upon insertion and withdrawal of the instrument 104, the position of the reference pattern 122 on the fixed surface 124 remains essentially constant during insertion and withdrawal of the instrument 104 into the sample 100.

The reference surface 124 can be any surface in the room or environment in which the procedure is carried out. For example, when the procedure occurs in a medical setting, the reference surface 124 can be a wall or ceiling, or a computerized tomograph (“CT”) or magnetic resonance imaging (“MRI”) gantry. The reference mark 128 can be any reference mark. Suitable reference marks 128 need not have been deliberately placed on the fixed surface 124. For example, the reference mark 128 can be a speck of dirt, a portion of an image or poster, or any other distinguishable feature of the fixed surface 124. Alternatively, the reference mark 128 can be a small black, white, or colored sticker that can be positioned randomly on the reference surface 124. The reference surface 124 need not be prepared specially to use the alignment system provided by the projected pattern 122. Thus, the instrument 104 that is coupled to the light source 120 can be used in any room or environment.

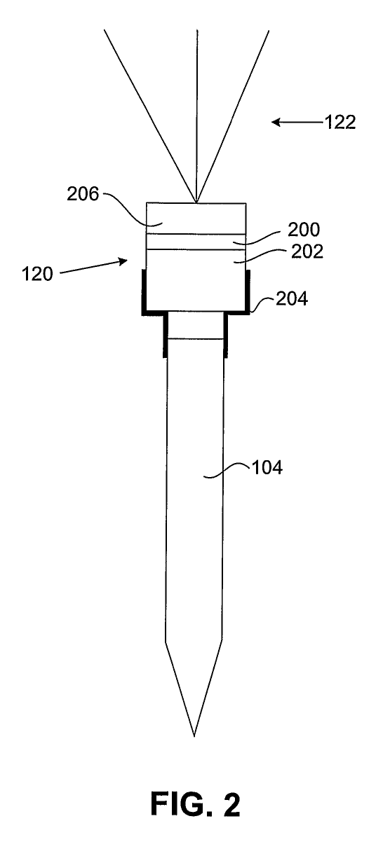

As shown in FIG. 2, one example of a light source 120 is a semiconductor laser 200 powered by a battery 202. The light source 120 is attached to the instrument 104, which can be, for example, a coaxial biopsy system or biopsy gun. The light source 120 can be integrated with the instrument 104 or releasably attached to the instrument 104 (e.g., to a common medical or other instrument) by an adaptor 204 (e.g., a Luer lock).

An optical system 206, which can include optical masks, filters, beam splitters, prisms, mirrors, and diffractive elements, can be included within the housing of the light source 120 to generate the reference pattern 122. Such elements can be customized to produce a desired reference pattern 122 and to direct that reference pattern 122 in a desired direction for projection onto a reference surface 124. That direction need not be one defined by the instrument 104. For example, the optical system 206 may include a moveable beam re-directing element, such as a mirror or prism, for projecting the reference pattern 122 against any convenient reference surface 124. Combinations of multiple optical elements in the optical system 206 can also be used to produce user-specified coordinate systems. Furthermore, different optical systems 206 can be removably inserted into the housing of the light source 120, so that a user can select a desired reference pattern 122. The optical system 206 remains in a fixed orientation with respect to the light source 120 during a single imaging and repositioning cycle of a procedure, and the light source 120 remains in a fixed orientation relative to the instrument 104 during a single imaging and repositioning cycle.

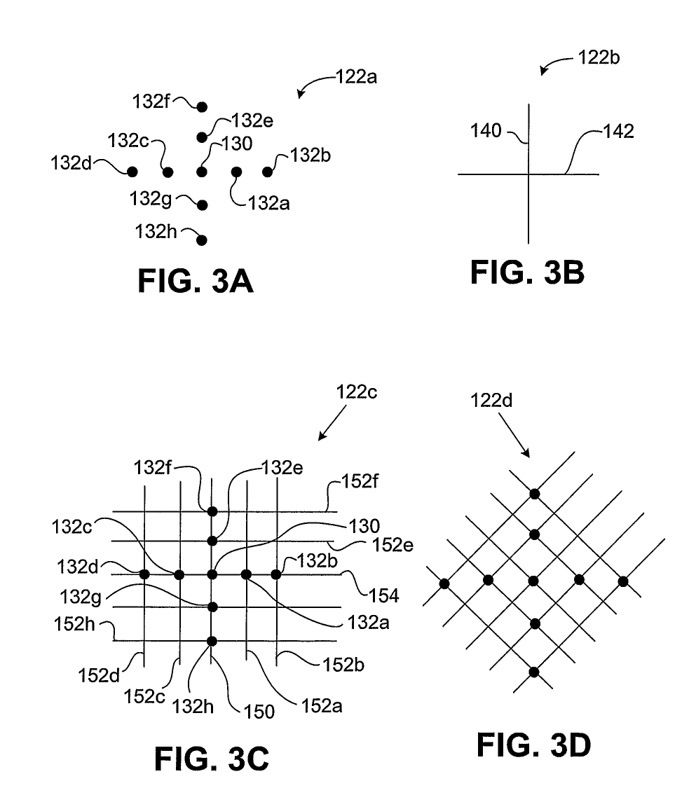

As shown in FIGS. 3A-E, various reference patterns 122a-122d can be projected from the light source 120 onto the fixed surface 124 for use as alignment aids. For example, as shown in FIG. 3A, a reference pattern can be a discrete crosshair pattern 122a defining a Cartesian coordinate system having an x-axis and a y-axis, each defined by dots extending away from a center dot 130 in directions that differ by 90 degrees. For example, the light beam that forms dot 132a can be emitted from the light source 120 in a direction that forms an angle of one degree in the positive x-direction with the beam that forms center dot 130. Similarly, dot 132b can represent an angle of positive two degrees along the x-direction; dot 132c can represent an angle of negative one degree along the x-direction; and dot 132d can represent an angle of negative two degrees. Along the y-axis, dot 132e can represent an angle of positive one degree; dot 132f can represent an angle of positive two degrees; dot 132g can represent an angle of negative one degree; and dot 132h can represent an angle of negative two degrees.

As shown in FIG. 3B, a reference pattern can be a continuous crosshair pattern 122b having a vertical line 140 and a horizontal line 142 projected onto the reference surface 124. The continuous crosshair pattern 122b can be generated by passing a laser beam through an optical system 20b having a diffractive optical element that spreads a light beam into two perpendicular planes. The optical system 206 can be rotated about the beam axis to rotate the orientation of the planes that make up the continuous crosshair pattern 122b.

As shown in FIG. 3C, a reference pattern can also be a rectilinear grid pattern 122c created by passing the nine beams used to form the discrete crosshair pattern 122a through the optical system 206 used to form the continuous crosshair pattern 122b. Doing so spreads each beam of the discrete crosshair pattern 122a into two perpendicular planes. When passed through the diffractive optical element, the beams 130, 132a, 132b, 132c, 132d that lie on the x-axis spread into planes that project vertical lines along the y-axis 150, 152a, 152b, 152c, 152d, respectively, onto the reference surface 124. Each beam 130, 132a, 132b, 132c, 132d that lies on the x-axis, when passed through the same optical system 206, also spreads the beam into a plane of light that projects a horizontal line 154 along the x-axis. Similarly, each linear beam 130, 132e, 132f, 132g, 132h that lies along the y-axis, when passed through the same optical system 206, projects a horizontal line 154, 152e, 152f, 152g, 152h, respectively and a vertical line 150 onto the reference surface 124.

As shown in FIG. 3D, rotating the diffractive optical element 45 degrees to the position of the optical system 206 used to create the rectilinear grid pattern 122c result in yet another reference pattern: a diagonal grid pattern 122d of perpendicular lines oriented at 45 with respect to the vertical and horizontal axis. The line spacing in the diagonal grid pattern 122d is smaller than the line spacing in the rectilinear grid pattern 122c by a factor of \sqrt{2}.

As shown in FIG. 3E, rotating the diffractive optical system 206 by an angle of arctan(2) degrees (i.e., 63.4 degrees) from the orientation of the optical system 206 used to create the rectilinear grid pattern 122c, results in a variable grid pattern 122e having a line spacing within the square defined by beams 132a, 132e, 132c, and 132g. Outside the square defined by beams 132a, 132e, 132c, and 132g, the line spacing of pattern 122e is twice the line spacing in the pattern 122c in one dimension and is equal to the line spacing in pattern 122c in an orthogonal direction. In general, when the optical element is rotated by an angle arctan(N), the separation angle R between lines in the pattern defined by a laser beam shining through the diffractive optical element is given by

tan(R)=(tan Y)/[(N²+1)(sin [arctan(1/N)])],

where Y is the angle of beam separation used to create the rectilinear grid pattern 122c.

Projected reference patterns 122a, 122b, 122c, 122d, and 122e can be used to align the instrument 104 within the sample 100, thereby enabling the instrument 104 to be guided towards a target 102 either within the sample 100 or on an opposite side of the sample 100 from an insertion site 106. The procedure for doing so includes determining the angle between the insertion site 106 and the target 102, then determining the angle of the instrument 104 in a partially inserted position. The deviation between the two angles is calculated. Then, the angle of the instrument 104 is adjusted until it aligns with the direction of a line extending between the insertion site 106 and the target 102.

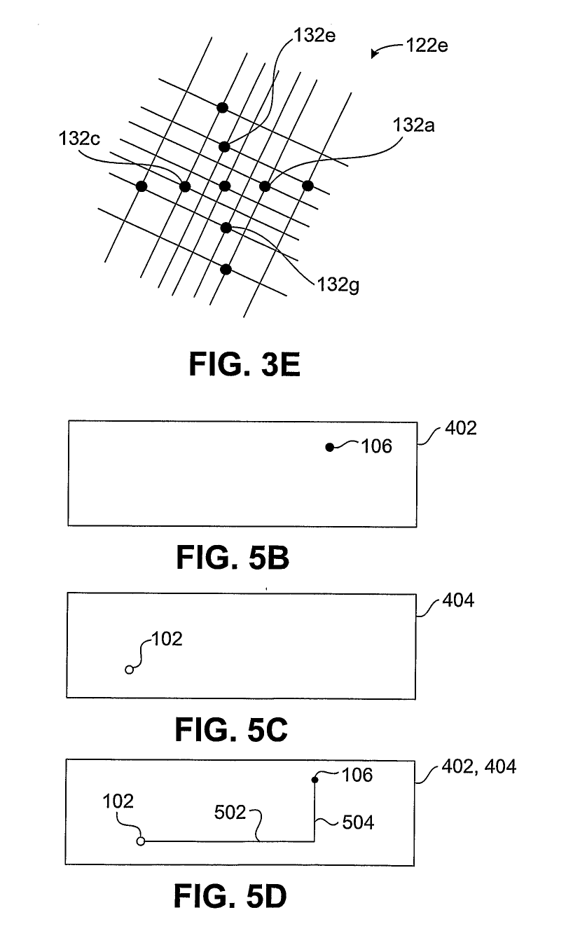

As shown in FIG. 4A, the instrument 104 is inserted into a sample 100 at the insertion site 106 on the surface of the sample 106 towards a target 102. While the instrument 104 is partially inserted into the sample 100, images of axial slices 402, 404, 406, 408, 410 can be recorded (e.g., with a CT scanner or with a MRI scanner). By examining images of axial slices that record the position of the insertion site 106, the instrument 104, and the position of the target 102, and by knowing the thickness of each axial slice, the angle φ, perpendicular to the plane of axial imaging, between a line from the insertion site 106 to the instrument tip 108 and a line from the insertion site 106 in the plane of the imaging slice (usually vertical or a known deviation from vertical) to the target 102 can be determined.

For example, FIG. 4B is a schematic two-dimensional representation of a first axial slice 402 from FIG. 4A that contains the insertion site 106, the target 102, and part of the instrument 104. FIG. 4C is a schematic two-dimensional representation of a second axial slice 404 from FIG. 4A containing a portion of the instrument 104. FIG. 4D is a schematic two-dimensional representation of a third axial slice 406 from FIG. 4A containing the tip of the instrument 104. By measuring the distance of the instrument 104 on the second axial slice 404, the angle between the line defined by the axis of the instrument 104, and a vertical, or near vertical in-plane line extending between the insertion site 106 and the target 102 can be determined. The tangent of the angle Xₒ (shown in FIG. 4A) is equal to the slice thickness divided by an in-slice measured length of the instrument 104. Additionally, if the instrument 104 traverses multiple axial slices, the tangent of angle Xₒ is equal to the combined distance divided by the product of the number of axial slices and the axial slice thickness.

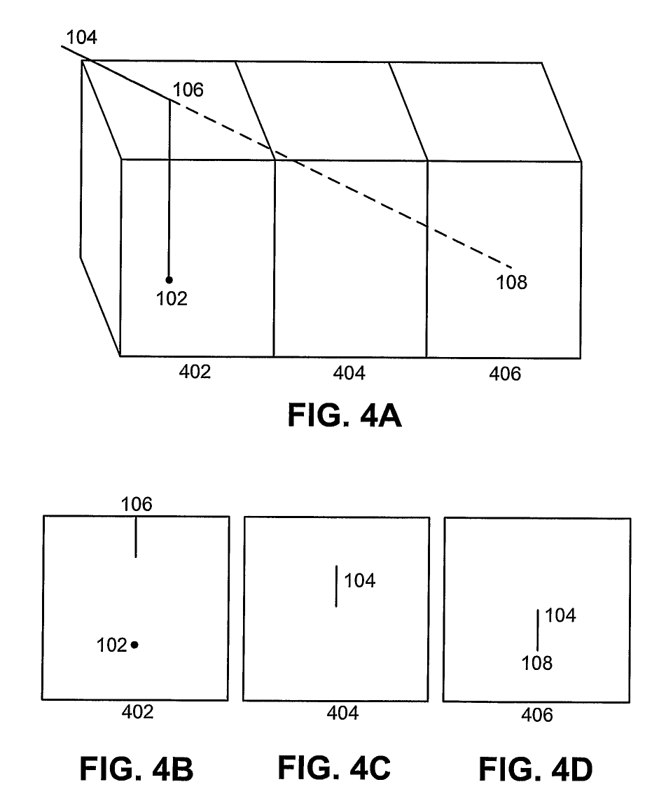

For example, the image of the first axial slice 402, including the position of the insertion point 106, is shown in FIG. 5B; the image of the second axial slice 404, including the position of the target 102; is shown in FIG. 5C; and an overlay of images of the first and second axial slices 402, 404 is shown in FIG. 5D. A parallel distance 502 between the insertion site 106 and the target 102, shown in FIG. 5D, is equal to the component of the distance from the insertion site 106 to the target 102 that is parallel to the front and top faces of the sample 100 as shown in FIG. 5A. The component of φ in a direction parallel to the parallel distance 502 is equal to the inverse tangent of the parallel distance 502 divided by a vertical distance between the insertion site 106 and the target 102 (i.e., the distance along a line perpendicular to the axial slices and to the top face of the sample 100 and extending between the insertion site 106 and the target 102). The vertical distance is determined by multiplying the thickness of each axial slice by the number of axial slices between the axial slices in which the insertion site 106 and the target 102 lie. The perpendicular distance 504 between the insertion site 106 and the target 102, shown in FIG. 5D, is equal to the component of the distance from the insertion site 106 to the target 102 that is perpendicular to the front face and parallel to the top face of the sample 100, as shown in FIG. 5A. The component of φ in a direction parallel to the perpendicular distance 504 is equal to the inverse tangent of the perpendicular distance 504 divided by the vertical distance between the insertion site 106 and the target 102.

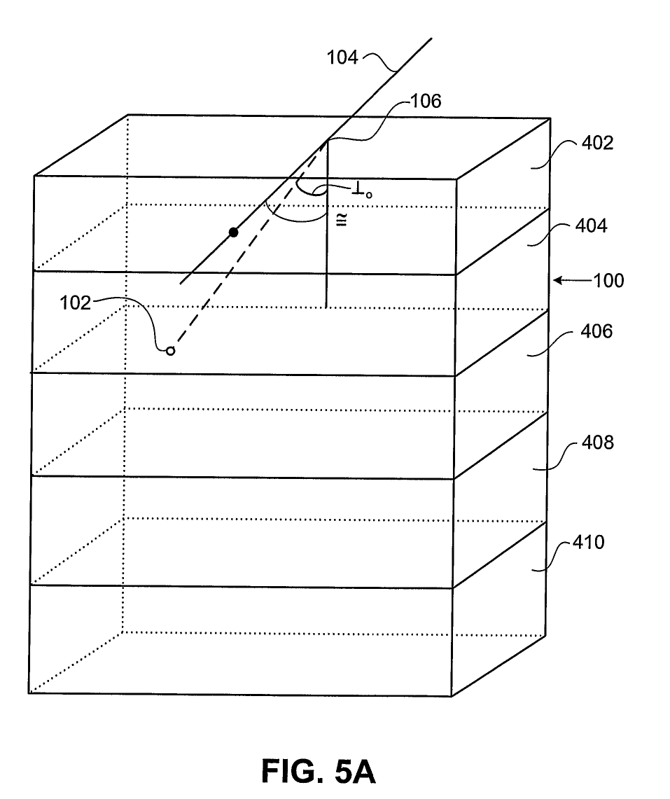

The angular orientation of the instrument 104 and the angle ψ (shown in FIG. 5A) that the longitudinal axis of the instrument 104 makes with a vertical line perpendicular to the top surface of the sample 100 can be determined from analysis of an image of an axial slice 402 through which the instrument 104 penetrates. For example, as shown in FIG. 6, an image of an axial slice 402 shows the insertion site 106 at which the instrument 104 enters the axial slice 402 and an exit site 602 at which the instrument 104 exits the axial slice 402. A parallel distance 604 is equal to a distance between the insertion site 106 and the exit site 602 along a line parallel to the front and top faces of the sample 100. A perpendicular distance 606 is equal to a distance between the insertion site 106 and the exit site 602 along a line perpendicular to the front face and parallel to the top face of the sample 100. The component of ψ in a direction parallel to the parallel distance 604 is equal to the inverse tangent of the parallel distance 604 divided by the thickness of the axial slice 402. The component of ψ in a direction parallel to the perpendicular distance 606 is equal to the inverse tangent of the perpendicular distance 606 divided by the thickness of the axial slice 402.

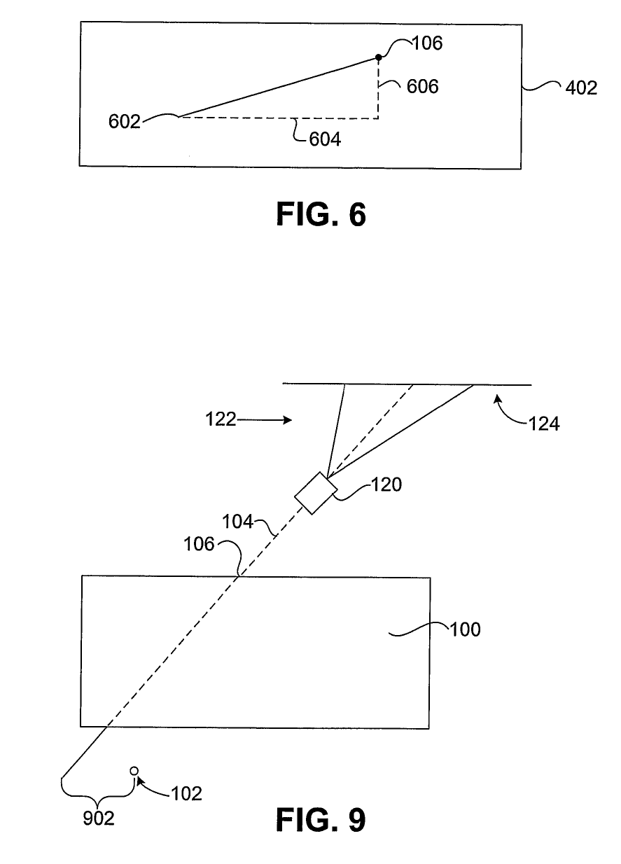

Once determined, the angular orientation of the instrument 104 is compared to the angle between the insertion site 106 and the target 102. The deviation of the instrument's orientation from the desired orientation is then measured by subtracting the angle ψ from the angle φ. By observing the position of the reference pattern 122 relative to the reference mark 128 on the reference surface 124, one then adjusts the orientation of the instrument 104 to align it with the desired orientation. For example, the parallel components of ψ and φ could differ by three degrees and the perpendicular components of ψ and φ could differ by one degree when the instrument 104 is in its misaligned position. Then, referring to FIG. 7, the position of the reference pattern 122e projected from the light source 120 onto the reference surface 124 (which includes the reference mark 128) can be used to adjust the orientation of the instrument 104. For example, if the reference mark 128 is positioned at the intersection of the lines 152e and 152c, and if each parallel line of the reference pattern 122c is emitted from the light source at angles that differ by one degree, the instrument 104 is repositioned such that the pattern is projected onto the reference surface 124 at a position in which the reference mark 128 lies at the intersection of lines 152e and 152f.

Once repositioned, or reoriented, the instrument 104 is imaged again to determine if it is now oriented along the line between the insertion site 106 and the target 102. If the angular orientation of the instrument 104 still differs from the desired orientation, it can be repositioned or reoriented again with the aid of the reference pattern 122 that is projected onto the reference surface 124.

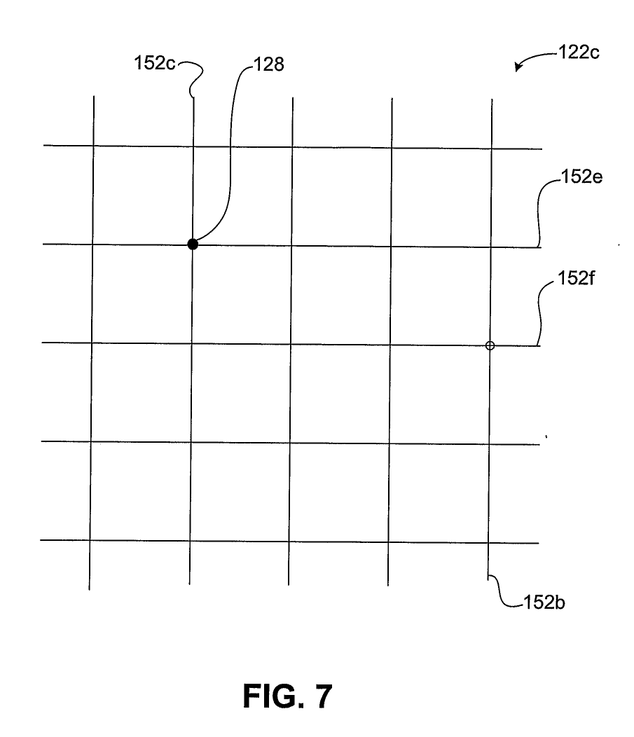

An instrument 104 that is relatively thin and flexible (e.g., a biopsy needle) can bend during the insertion procedure. This bending can cause an inaccurate measurement of the orientation of the instrument's longitudinal axis within the sample 100. To compensate for this error, a rigid instrument guide 802 (see FIG. 8) is used to guide the instrument along an axis. In some embodiments, the instrument guide 802 is a tube of rigid material having longitudinal holes of different diameters 804 through which instruments 104 (e.g., needles) of different sizes are passed to enter the sample 100 through the insertion site 106. The instrument guide 802 can be rigidly attached to a housing 806 for the light source 120, such that when the angular orientation of the instrument 104 changes, the orientation of the reference pattern 122 projected from the light source 120 changes by a comparable amount. The instrument guide 802 can also be connected to the housing 806 so that their respective longitudinal axes are parallel, rather than at an angle as shown in FIG. 8. In some embodiments, the instrument guide 802 is be configured to connect to a tool or instrument that is larger than the guide 802. In addition, the housing 806 can be attached to the top of the instrument or tool guide (with the hence longitudinal axes of the guide and the housing aligned). This allows the housing 806, and the light source 120, to be used interchangeably with various tools or instruments. Furthermore, the instrument guide 802 can be integrally formed with the housing 806 or can be detachably coupled to the housing 806, for example, with a snap-fit mechanical coupling.

The instrument guide 802 may remain at the insertion site 106 while the instrument 104 moves to and from the target 102. When the instrument guide 802 remains at the insertion site 106, the weight of the light source 120 acts through a shorter torque arm and thereby exerts less torque on the instrument 104. Other designs can also reduce the torque on the instrument 104. For example, one can use a lighter material. Alternatively, one can connect the power source or battery 202 to the light source 120 by fine wires instead of mounting it on the light source 120.

In addition to being used to align an instrument 104 as it advances toward a target 102, the reference pattern 122 can also be used to detect patient movement during manipulation, sampling, treatment, or subsequent imaging or procedures. For example, when the sample 100 is tissue in a patient and the light source 120 is positioned on the tissue 100, as shown in FIG. 8, the movement of the reference pattern 122 projected onto the surface 124 indicates patient movement. The movement of the projected reference pattern 122 on the surface 124 can be used to reposition the tissue 100 of the patient (or the entire patient) in an original position (for example, for a subsequent procedure).

Movement of the projected reference pattern 122 also reveals any movement caused by a periodic physiological process. For example, when the integrated instrument guide 802 and light source housing 806 are positioned on the skin of a patient 100 and an instrument 104 is inserted into the patient, the patient's breathing or heart beat causes the instrument's orientation to oscillate between two positions. Axial slice images of the patient 100 created at a particular phase of the patient's pulmonary or cardiac cycle permit comparison between the direction from the insertion site 106 to the target site 102 and the orientation of the instrument 104, as indicated by the position of the projected pattern 122 at a particular phase of its oscillation.

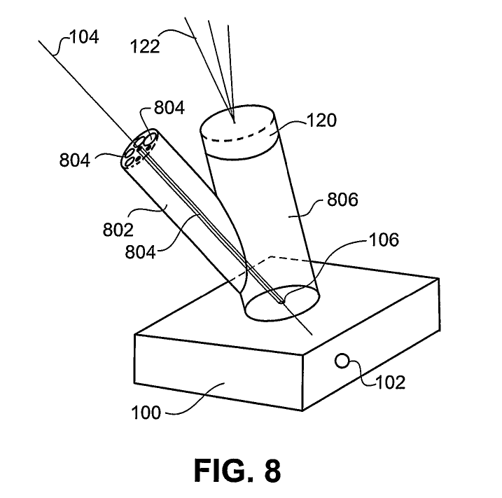

As shown in FIG. 9, the light source 120 can also be used to align the instrument 104 so that it reaches a target site 102 on a side of the sample 100 opposite the insertion site 106. The instrument 104 (e.g., a drill) can be inserted entirely through the sample 100 (e.g., a wall, a board, a floor) from an insertion site 106 in the general direction of the target site 102. When the instrument 104 emerges from the sample 100, a horizontal distance 902 between it and the target site 102 can be measured. The angular deviation of the longitudinal direction of the instrument 104 from the line connecting the insertion site 106 and the target site 102 can then be determined. The projected reference pattern 122 can then be used to realign the instrument 104 such that, when reinserted through the sample 100, it reaches the target site 102.

It is to be understood that while the invention has been described in conjunction with the detailed description thereof, the foregoing description is intended to illustrate and not to limit the scope of the invention, which is defined by the scope of the appended claims. Other aspects, advantages, and modifications are within the scope of the following claims:

1. A method of adjusting an orientation of an apparatus relative to a surface of a sample, the method comprising:
positioning the apparatus in an initial orientation relative to the surface;
projecting a reference pattern from the apparatus onto a reference surface, the position of the projected reference pattern on the reference surface being responsive to a change in an angular orientation of the apparatus relative to the initial orientation;
on the basis of a position of the projected reference pattern, determining an angular deviation of the apparatus from a desired orientation; and
adjusting the orientation of the apparatus, such that the position of the reference pattern projected on the reference surface indicates a reduction in the angular deviation.

2. The method of claim 1, wherein projecting a reference pattern comprises projecting a ring that moves in response to a change in an angular orientation of the apparatus relative to the initial orientation.

3. The method of claim 1, wherein projecting a reference pattern comprises projecting lines that move in response to a change in an angular orientation of the apparatus relative to the initial orientation.

4. The method of claim 1, wherein positioning the apparatus comprises positioning a biopsy needle.

5. The method of claim 1, wherein projecting a reference pattern comprises projecting a first beam emitted from the apparatus and a second beam emitted from the apparatus at a predetermined angle relative to the first beam.

6. The method of claim 1, further comprising:
inserting the apparatus into the sample; and
while the apparatus is inserted, imaging the sample and the apparatus to determine the angular deviation.

7. The method of claim 6, further comprising:
withdrawing the apparatus, at least partially, from the sample; and
re-inserting the apparatus into the sample such that the angular deviation is reduced.

8. The method of claim 7, wherein positioning the apparatus comprises positioning a biopsy needle.

9. The method of claim 6, wherein imaging the sample and the apparatus comprises separately imaging a plurality of axial slices of the sample.

10. The method of claim 9, further comprising imaging each of the axial slices at substantially the same phase of a periodic physiological process.

11. The method of claim 9, further comprising imaging each of the axial slices at substantially the same phase of a pulmonary cycle.

12. The method of claim 9, further comprising imaging each of the axial slices at substantially the same phase of a cardiac cycle.

13. An apparatus comprising:
an instrument;
a light source adapted for coupling to the instrument; and
an optical system positioned along a path of light emitted from the light source, the optical system being adapted to transform light emitted from the light source into a reference pattern that defines a coordinate system, and to project the reference pattern on a reference surface.

14. The apparatus of claim 13, wherein the instrument comprises a medical instrument.

15. The apparatus of claim 13, wherein the instrument comprises a biopsy needle.

16. The apparatus of claim 13, wherein the optical element is adapted to include, in the reference pattern, a feature identifying an angular orientation of the instrument.

17. The apparatus of claim 13, wherein the optical element is adapted to include, in the reference pattern, a ring identifying an angular orientation of the medical instrument.

18. The apparatus of claim 13, wherein the optical element is adapted to include, in the reference pattern, lines identifying an angular orientation of the instrument.

19. The apparatus of claim 13, wherein the optical element is adapted to a first beam and a second beam, the second beam being oriented at a predetermined angle relative to the first beam.

20. The apparatus of claim 13, wherein the light source comprises a laser.

21. The apparatus of claim 13, wherein the light source comprises a light-emitting diode.

22. The apparatus of claim 13, wherein the light source is oriented to emit light in a direction that differs from a direction defined by the instrument.

23. The apparatus of claim 13, further comprising an instrument guide adapted for guiding the instrument along an axis.

24. An apparatus for adjusting the orientation of an instrument that is adapted to be inserted into a sample, the apparatus comprising:
an instrument guide adapted for guiding the instrument along an axis.
a light source coupled to the instrument guide; and
an optical system positioned in a path of light emitted from the light source, the optical system being adapted to transform light emitted from the light source into a reference pattern that defines a coordinate system, and to project the reference pattern onto a reference surface.

25. The apparatus of claim 24, wherein the optical system is adapted to project light in a direction that differs from a direction defined by a longitudinal axis of the instrument guide.

26. The apparatus of claim 24, wherein the instrument guide is detachably coupled to the light source.

27. The apparatus of claim 24, wherein the instrument guide comprises a tube for guiding the instrument.
